(19) United States
(12) Patent Application Publication
(10) Pub. No.: US 2009/0069666 A1
(43) Pub. Date: Mar. 12, 2009

(54) Correction of Intensity Inhomogeneity in Breast MRI

(75) Inventors: Gerardo Hermosillo Valadez, West Chester, PA (US); Yoshihisa Shinagawa, Downingtown, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/205,220

(22) Filed: Sep. 5, 2008

Related U.S. Application Data
(60) Provisional application No. 60/971,318, filed on Sep. 11, 2007.

(51) Int. Cl. A61B 5/055; A61B 5/00

(52) U.S. Cl. 600/410; 382/131

(57) ABSTRACT

A method for correcting for magnetic field inhomogeneity in a breast MRI, includes acquiring a magnetic resonance (MR) image including a patients breast. A surface image of the breast is automatically isolated within the acquired MR image based on a high contrast between the breast and surrounding air. A multiplicative field that transforms the isolated surface image into a uniform intensity is generated. The generated multiplicative field is applied to the entire acquired MR image to produce a corrected image.

### CROSS-REFERENCE TO RELATED APPLICATION

The present application is based on provisional application Ser. No. 60/971,318 filed Sep. 11, 2007, the entire contents of which are herein incorporated by reference.

### BACKGROUND OF THE INVENTION

1. Technical Field

The present disclosure relates to breast MRI and, more specifically, to correction of intensity in homogeneity in breast MRI.

2. Discussion of Related Art

Computer aided diagnosis (CAD) is the process of using computer vision systems to analyze medical image data and make a determination as to what regions of the image data are potentially problematic. Some CAD techniques then present these regions of suspicion to a medical professional such as a radiologist for manual review, while other CAD techniques make a preliminary determination as to the nature of the region of suspicion. For example, some CAD techniques may characterize each region of suspicion as a lesion or a non-lesion. The final results of the CAD system may then be used by the medical professional to aid in rendering a final diagnosis.

Because CAD techniques may identify lesions that may have been overlooked by a medical professional working without the aid of a CAD system, and because CAD systems can quickly direct the focus of a medical professional to the regions most likely to be of diagnostic interest, CAD systems may be highly effective in increasing the accuracy of a diagnosis and decreasing the time needed to render diagnosis. Accordingly, scarce medical resources may be used to benefit a greater number of patients with high efficiency and accuracy.

CAD techniques have been applied to the field of mammography, where low-dose x-rays are used to image a patient's breast to diagnose suspicious breast lesions. However, because mammography relies on x-ray imaging, mammography may expose a patient to potentially harmful ionizing radiation. As many patients are instructed to undergo mammography on a regular basis, the administered ionizing radiation may, over time, pose a risk to the patient. Moreover, it may be difficult to use x-rays to differentiate between different forms of masses that may be present in the patient's breast. For example, it may be difficult to distinguish between calcifications and malignant lesions.

Magnetic resonance imaging (MRI) is a medical imaging technique that uses a powerful magnetic field to image the internal structure and certain functionality of the human body. MRI is particularly suited for imaging soft tissue structures and is thus highly useful in the field of oncology for the detection of lesions.

In dynamic contrast enhanced MRI (DCE-MRI), many additional details pertaining to bodily soft tissue may be observed. These details may be used to further aid in diagnosis and treatment of detected lesions.

DCE-MRI may be performed by acquiring a sequence of MR images that span a time before magnetic contrast agents are introduced into the patient's body and a time after the magnetic contrast agents are introduced. For example, a first MR image may be acquired prior to the introduction of the magnetic contrast agents, and subsequent MR images may be taken at a rate of one image per minute for a desired length of time. By imaging the body in this way, a set of images may be acquired that illustrate how the magnetic contrast agent is absorbed and washed out from various portions of the patient's body. This absorption and washout information may be used to characterize various internal structures within the body and may provide additional diagnostic information.

It may be particularly difficult or impossible to ensure a fully-homogeneous magnetic field when acquiring MR images. Accordingly, there are often subtle variations in the strength of the applied magnetic field with respect to location. For example, the applied magnetic field may be stronger in one location and weaker in another location. The applied magnetic field may also vary with respect to time. For example, in the sequence of DCE-MR images, the variations in the magnetic field may change from image to image.

These variations may have an impact on the resulting image quality and may lead to the introduction of artifacts. Additionally, the level of enhancement in the post-contrast image sequence may be affected. The effect of the inhomogenious magnetic field may be to have uneven enhancement throughout portions of the image that should appear uniform. This unevenness may adversely effect segmentation of the breast from the remainder of the image data because in segmentation, it is often assumed that a particular organ appears homogeneously.

### SUMMARY

A method for correcting for magnetic field inhomogeneity in a breast MRI includes acquiring a magnetic resonance (MR) image including a patient's breast. A surface image of the breast is automatically isolated within the acquired MR image based on a high contrast between the breast and surrounding air. A multiplicative field that transforms the isolated surface image into a uniform intensity is generated. The generated multiplicative field is applied to the entire acquired MR image to produce a corrected image.

The breast may then be segmented from the corrected image and one or more regions of suspicion may be automatically identified from the segmented breast. Then each of the identified regions of suspicion may be determined to be false positives or actual lesions. The location of the determined actual lesions may be presented to a user.

The acquired MR image may be a dynamic contrast enhanced MRI including a pre-contrast MR image and a sequence of post-contrast MR images acquired at a regular interval of time after administration of a magnetic contrast agent.

The acquired MR image may be a dynamic contrast enhanced MRI including a pre-contrast MR image and a sequence of post-contrast MR images acquired at a regular interval of time after administration of a magnetic contrast agent. The automatic identification of the regions of suspicion may include identifying the regions of suspicion based on an absorption and washout profile observed from the sequence of post-contrast MR images.

One or more of the identified regions of interest may be automatically characterized according to a BIRADS classification based on the absorption and washout profile for the respective identified region of suspicion observed from the sequence of post-contrast MR images.

The surface image of the breast may include a set of image voxels of the acquired MR image that are in contact with the surrounding air. The surface image of the breast may also include image voxels of the acquired MR image that are within a predetermined depth from the image voxels of the acquired MR image that are in contact with the surrounding air. The surface image of the breast may be an image of the breast skin.

A method for automatically detecting breast lesions includes receiving a dynamic contrast enhanced magnetic resonance image (DCE-MRI) including a patient's breast. A surface image of the breast is automatically isolated within the received DCE-MRI based on a high contrast between the breast and surrounding air. A corrected image may be produced from the DCE-MRI based on an intensity distribution of the isolated surface image.

The production of the corrected image may include generating a multiplicative field that transforms the isolated surface image into a uniform intensity and applying the generated multiplicative field to the entire acquired MR image to produce a corrected image.

The breast may be segmented from the corrected image. One or more regions of suspicion may be automatically identified from the segmented breast. Each of the identified regions of suspicion may be determined to be either a false positive or an actual lesion. The location of the determined actual lesions may be presented to a user.

The surface image of the breast may include a set of image voxels of the acquired MR image that are in contact with the surrounding air. The surface image of the breast may also include image voxels of the acquired MR image that are within a predetermined depth from the image voxels of the acquired MR image that are in contact with the surrounding air. The surface image of the breast may be an image of the breast skin.

A computer system includes a processor and a program storage device readable by the computer system, embodying a program of instructions executable by the processor to perform method steps for correcting for magnetic field inhomogeneity in a breast MRI. The method includes acquiring a magnetic resonance (MR) image including a patient's breast. A surface image of the breast is automatically isolated within the acquired MR image based on a high contrast between the breast and surrounding air. A multiplicative field that transforms the isolated surface image into a uniform intensity is generated. The generated multiplicative field is applied to the entire acquired MR image to produce a corrected image.

The breast may then be segmented from the corrected image and one or more regions of suspicion may be automatically identifying from the segmented breast. The identified regions of suspicion may then be determined to be either a false positive or an actual lesion. The location of the determined actual lesions may be presented to a user.

The surface image of the breast may include a set of image voxels of the acquired MR image that are in contact with the surrounding air. The surface image of the breast may further include image voxels of the acquired MR image that are within a predetermined depth from the image voxels of the acquired MR image that are in contact with the surrounding air. The surface image of the breast may be an image of the breast skin.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a flow chart illustrating a method for imaging a patient's breast using DCE-MRI and rendering a computer-aided diagnosis according to an exemplary embodiment of the present invention;

FIG. 2 is a set of graphs illustrating a correspondence between absorption and washout profiles for various BIRADS classifications according to an exemplary embodiment of the present invention;

FIG. 3 is a flow chart illustrating a method for correcting for inhomogeneity in the applied magnetic field according to an exemplary embodiment of the present invention; and

FIG. 4 shows an example of a computer system capable of implementing the method and apparatus according to embodiments of the present disclosure.

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Exemplary embodiments of the present invention seek to image a patient's breast using DCE-MRI techniques and then perform CAD to identify regions of suspicion that are more likely to be malignant breast lesions. By utilizing DCE-MRI rather than mammography, additional data pertaining to contrast absorption and washout may be used to accurately distinguish between benign and malignant breast masses.

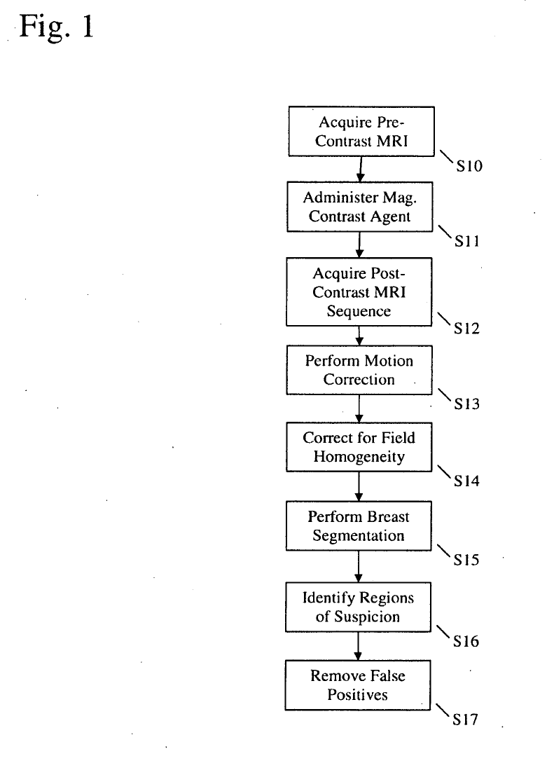

FIG. 1 is a flow chart illustrating a method for imaging a patient's breast using DCE-MRI and rendering a computer-aided diagnosis according to an exemplary embodiment of the present invention. First, a pre-contrast MRI is acquired (Step S10). The pre-contrast MRI may include an MR image taken of the patient before the magnetic contrast agent has been administered. The pre-contrast MRI may include one or more modalities. For example, both T1 and T2 relaxation modalities may be acquired.

Next, with the patient remaining as still as possible, the magnetic contrast agent may be administered (Step S11). The magnetic contrast agent may be a paramagnetic agent, for example, a gadolinium compound. The agent may be administered orally, intravenously, or by another means. The magnetic contrast agent may be selected for its ability to appear extremely bright when imaged in the T1 modality. By injecting the magnetic contrast agent into the patient's blood, vascular tissue may be highly visible in the MRI. Because malignant tumors tend to be highly vascularized, the use of the magnetic contrast agent may be highly effective for identifying regions suspected of being lesions.

Moreover, additional information may be gleamed by analyzing the way in which a region absorbs and washes out the magnetic contrast agent. For this reason, a sequence of post-contrast MR images may be acquired (Step S12). The sequence may be acquired at regular intervals in time, for example, a new image may be acquired every minute.

As discussed above, the patient may be instructed to remain as still as possible throughout the entire image acquisition sequence. Despite these instructions, the patient will most likely move somewhat from image to image. Accordingly, before regions of suspicion are identified (Step S16), motion correction may be performed on the images (Step S13).

At each acquisition, the image may be taken in the T1 modality that is well suited for monitoring the absorption and washout of the magnetic contrast agent.

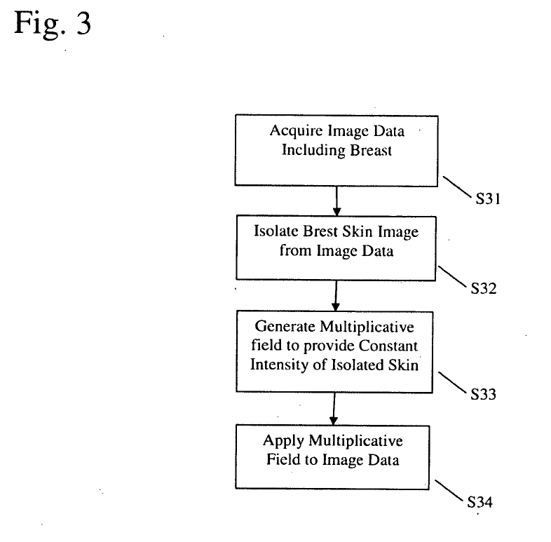

As discussed above, because MR images are acquired using a powerful magnetic field, subtle inhomogeneity in the magnetic field may have an impact on the image quality and may lead to the introduction of artifacts. Additionally, the level of enhancement in the post-contrast image sequence may be affected. Also, segmentation of the breast may be impeded by the inhomogeneity, as in segmentation, it is often assumed that a particular organ appears homogeneously. Accordingly, the effects of the inhomogeneous magnetic field may be corrected for within all of the acquired MR images (Step S14). A process for correcting for inhomogeneity in the applied magnetic field according to an exemplary embodiment of the present invention is discussed in detail below with respect to FIG. 3.

The order in which motion correction (Step S13) and inhomogeneity correction (Step S14) are performed on the MR images is not critical. All that is required is that these steps be performed after image acquisitions for each given image, and prior to segmentation (Step S15). These corrective steps may be performed for each image after each image is acquired or for all images after all images have been acquired.

After the corrective steps (Steps S13 and S14) have been performed, breast segmentation may be performed (Step S15). Segmentation is the process of determining the contour delineating a region of interest from the remainder of the image. In making this determination, edge information and shape information may be considered.

Edge information pertains to the image intensity changes between the interior and exterior of the contour. Shape information pertains to the probable shape of the contour given the nature of the region of interest being segmented. Some techniques for segmentation such as the classical watershed transformation rely entirely on edge information. Examples of this technique may be found in L. Vincent and P. Soille, “Watersheds in digital spaces: An efficient algorithm based immersion simulations” IEEE Trans. PAMI, 13(6):583-589, 1991, which is incorporated by reference. Other techniques for segmentation rely entirely on shape information. For example, in M. Kass, A. Witkin, and D. Terzopoulous, “Snakes—Active contour models” Int J. Comp Vis, 1(4): 321-331, 1987, which is incorporated by reference, a calculated internal energy of the curvature is regarded as a shape prior although its weight is hard-coded and not learned through training. In A. Tsai, A. Yezzi, W. Wells, C. Tempany, D. Tucker, A. Fan, and W. E. Grimson, “A shape-based approach to the segmentation of medical imagery using level sets” IEEE Trans. Medical Imaging, 22(2): 137-154, 2003, which is incorporated by reference, the shape prior of signed distance representations called eigenshapes is extracted by Principal Component Analysis (PCA). When the boundary of an object is unclear and or noisy, the shape prior is used to obtain plausible delineation.

When searching for lesions in the breast using DCE-MRI, internal structures such as the pectoral muscles that are highly vascularized may light up with the application of the magnetic contrast agent. Thus, the pectoral muscles, and other such structures may make location of breast lesions more difficult. Accordingly, by performing accurate segmentation, vascularized structures that are not associated with the breast tissue may be removed from consideration thereby facilitating fast and accurate detection of breast lesions.

After segmentation has been performed (Step S15), the breast tissue may be isolated and regions of suspicion may be automatically identified within the breast tissue region (Step S16). A region of suspicion is a structure that has been determined to exhibit one or more properties that make it more likely to be a breast lesion than the regions of the breast tissue that are not determined to be regions of suspicion. Detection of the region of suspicion may be performed by systematically analyzing a neighborhood of voxels around each voxel of the image data to determine whether or not the voxel should be considered part of a region of suspicion. This determination may be made based on the acquired pre-contrast MR image as well as the post-contrast MR image. Such factors as size and shape may be considered.

Moreover, the absorption and washout profile of a given region may be used to determine whether the region is suspicious. This is because malignant tumors tend to show a rapid absorption followed by a rapid washout. This and other absorption and washout profiles can provide significant diagnostic information.

Breast imaging reporting and data systems (BIRADS) is a system that has been designed to classify regions of suspicion that have been manually detected using conventional breast lesion detection techniques such as mammography and breast ultrasound. Under this approach, there are six categories of suspicious regions. Category 0 indicates an incomplete assessment. If there is insufficient data to accurately characterize a region, the region may be assigned to category 0. A classification as category 0 generally implies that further imaging is necessary. Category 1 indicates normal healthy breast tissue. Category 2 indicates benign or negative. In this category, any detected masses such as cysts or fibroadenomas are determined to be benign. Category 3 indicates that a region is probably benign, but additional monitoring is recommended. Category 4 indicates a possible malignancy. In this category, there are suspicious lesions, masses or calcifications and a biopsy is recommended. Category 5 indicates that there are masses with an appearance of cancer and biopsy is necessary to complete the diagnosis. Category 6 is a malignancy that has been confirmed through biopsy.

Exemplary embodiments of the present invention may be able to characterize a given region according to the above BIRADS classifications based on the DCE-MRI data. To perform this categorization, the absorption and washout profile, as gathered from the post-contrast MRI sequence, for each given region may be compared against a predetermined understanding of absorption and washout profiles.

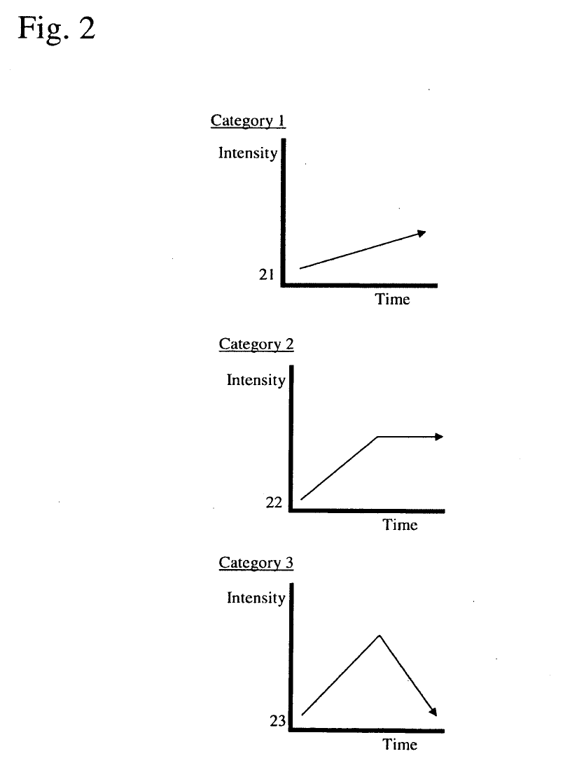

FIG. 2 is a set of graphs illustrating a correspondence between absorption and washout profiles for various BIRADS classifications according to an exemplary embodiment of the present invention. In the first graph 21, the T1 intensity is shown to increase over time with little to no decrease during the observed period. This behavior may correspond to a gradual or moderate absorption with a slow washout. This may be characteristic of normal breast tissue and accordingly, regions exhibiting this profile may be classified as category 1.

In the next graph 22, the T1 intensity is shown to increase moderately and then substantially plateau. This behavior may correspond to a moderate to rapid absorption followed by a slow washout. This may characterize normal breast tissue or a benign mass and accordingly, regions exhibiting this profile may be classified as category 2.

In the next graph 23, the T1 intensity is shown to increase rapidly and then decrease rapidly. This behavior may correspond to a rapid absorption followed by a rapid washout. While this behavior may not establish a malignancy, it may raise enough suspicion to warrant a biopsy, accordingly, regions exhibiting this profile may be classified as category 3.

Other absorption and washout profiles may be similarly established for other BIRAD categories. In this way, DCE-MRI data may be used to characterize a given region according to the BIRADS classifications. This and potentially other criteria, such as size and shape, may thus be used to identify regions of suspicion (Step S16).

After regions of suspicion have been identified, false positives may be removed (Step S17). As described above, artifacts such as motion compensation artifacts, artifacts cause by magnetic field inhomogeneity, and other artifacts, may lead to the inclusion of one or more false positives. Exemplary embodiments of the present invention and or conventional approaches may be used to reduce the number of regions of suspicion that have been identified due to an artifact, and thus false positives may be removed. Removal of false positives may be performed by systematically reviewing each region of suspicion multiple times, each time for the purposes of removing a particular type of false positive. Each particular type of false positive may be removed using an approach specifically tailored to the characteristics of that form of false positive. Examples of such approaches are discussed in detail below.

After false positives have been removed (Step S17), the remaining regions of suspicion may be presented to the medical practitioner for further review and consideration. For example, the remaining regions of interest may be highlighted within a representation of the medical image data. Quantitative data such as size and shape measurements and or BIRADS classifications may be presented to the medical practitioner along with the highlighted image data. The presented data may then be used to determine a further course of testing or treatment. For example, the medical practitioner may use the presented data to order a biopsy or refer the patient to an oncologist for treatment.

As discussed above, prior to performing breast segmentation, the acquired image data may be corrected for inhomogeneity of the magnetic field used in acquiring the MR image data. Exemplary embodiments of the present invention seek to correct for inhomogeneity in the applied magnetic field so that subsequent segmentation of the image data (for example, to segment the breast as is done in Step S15 discussed above) may be performed quickly and accurately.

Inhomogeneity may be corrected for by applying a multiplicative field to the image data. A multiplicative field is a mathematical expression that dictates how the intensity of each image voxel should be adjusted to obtain the desired inhomogeneity-compensated image data. The multiplicative field, once calculated, may be used to transform from the original image, which was acquired using an inhomogenious magnetic field, to a compensated image, which approximates as closely as possible, what the original image would be like if the applied magnetic field was fully homogeneous.

The multiplicative field may be calculated by identifying a reference portion of the acquired image that should ideally be of a uniform enhancement, but is non-uniform in the image data. It may then be assumed that the non-uniformity is the direct result of the inhomogeneity of the magnetic field that was applied during image acquisition, and thus the multiplicative field may be calculated by working backwards from the actual field to the ideal uniform field.

Thus the calculation of the multiplicative field relies on the assumption that the same function that can transform the uneven enhancement of the identified reference portion into the uniform ideal may also be used to transform the totality of the image data into an ideal version of the image data. In order for this assumption to be useful, the reference portion must be large enough to be included throughout the image data. If the reference portion is only included in a small section of the image data, it might not be beneficial to extrapolate the entire multiplicative field from the inhomogeneity of the small section of image data.

Moreover, the reference portion should be relatively easy to segment from the remainder of the image. If the reference portion is difficult to segment from the remainder of the image, then it is more likely that a section of image data that should not be part of the reference portion inadvertently becomes part of the segmentation. Also, if the reference portion is difficult to segment from the remainder of the image, then it is more likely that a section of the image data that should be part of the reference portion is indivertibly left out of the segmentation. Either error could potentially compromise the effectiveness of the calculated multiplicative field and could even result in a multiplicative field that makes ultimate segmentation of the image data even more difficult and prone to error than if no multiplicative field was used. Thus, if the reference portion is improperly segmented, correction of the image data using the calculated multiplicative field may actually make matters worse.

Exemplary embodiments of the present invention use the isolated breast skin within the image data as the reference portion from which the multiplicative field may be calculated. The breast skin provides a surface that may be quickly segmented from the image data with a high degree of accuracy owing to the high contrast that may be found in the image data between the breast skin and the air around it. This high contrast ensures that the breast skin may be accurately segmented even when magnetic field inhomogeneity is so profound that the level of enhancement between various areas of the breast skin within the image data vary widely. Moreover, the breast skin may be found throughout a large portion of the MR image data thus supporting the assumption that the multiplicative field that is found to even out the intensity of the reference portion may be fairly applied to the entire image data to obtain corrected image data that is more accurate than the original image data and is a fair estimation of what the image data would have looked like if the applied magnetic field were homogeneous.

The MR images may be modeled as functions from a subset of three-dimensional space Ω⊂ [symbol] ³ into the set of real numbers [symbol] or a subset thereof. The segmentation of the skin may produce a binary image S: Ω→{0,1} defined as:

$$S ( x ) = \{ \begin{matrix}1 & if x is a location on the skin , \\ 0 & otherwise .\end{matrix} \qquad ( 1 )$$

The multiplicative field φεℑ from an appropriate family ℑ of smooth functions may be applied to the original image I: Ω→ [symbol] to produce the corrected image C_{φ}(x)→ [symbol] :

C_{φ}(x)≡I(x)φ(x) (2)

The average intensity of the corrected image for locations on the skin (\tilde{C}_{φ}) may be given by:

\tilde{C}_{φ}≡∫_{Ω}S(x)C_{φ}(x)dx=∫_{Ω}S(x)I(x)φ(x)dx (3)

Given a field φ, the deviation D_{φ} from this average value may be measured as:

D_{φ}≡∫_{Ω}(S(x)C_{φ}(x)−\tilde{C}_{φ})²dx=∫_{Ω}(S(x)I(x)φ(x)−\tilde{C}_{φ})²dx (4)

The fact that the corrected image should have constant value at the skin is expressed by the fact that D_{φ} should be minimal. Accordingly, the optimal field φ* may be defined as the solution of the minimization problem:

$${φ}^{*} = arg {min}_{φ ∈} {D}_{φ} \qquad ( 5 )$$

An optimization algorithm may be employed to find a local solution to the minimization problem of equation 5 given an initial estimate φ₀.

FIG. 3 is a flow chart illustrating a method for correcting for inhomogeneity in the applied magnetic field according to an exemplary embodiment of the present invention. First, MR images including the breast may be acquired (Step S31). The MR images may be either a pre-contrast MR image or a post-contrast MR images, for example, an image from a DCE-MRI sequence.

Next, the breast skin may be identified within the image data and isolated from the remainder of the image data (Step S32). Isolation of the breast skin may include removal of all image data that is not breast skin. The breast skin may be quickly and accurately isolated by identifying the border of sharp contrast between the skin and the air around it. It is not necessary that the delineation between the breast skin and the breast tissue be determined, it may be presumed that the skin extends with a known thickness. Alternatively, it may be sufficient to remove all but the outer most voxel layer of the breast skin, as this data may be sufficient to calculate the multiplicative field.

After the breast skin has been isolated (Step S32), a multiplicative field may be generated and optimized such that the application of the multiplicative field to the isolated breast skin image data produces image data of uniform intensity throughout (Step S33). Finally, the generated multiplicative field may be applied to the entirety of the image data to produce a corrected image data (Step S34). The corrected image data may then be used to perform segmentation of the breast tissue, for example, as described above.

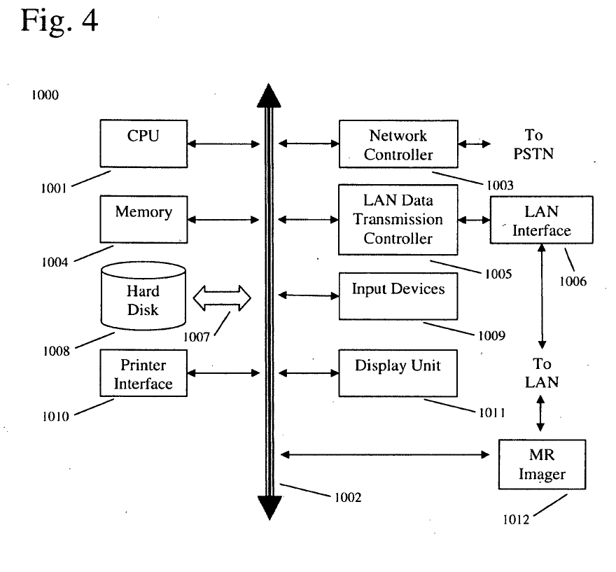

FIG. 4 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007. A MR imager 1012 may be connected to the internal bus 1002 via an external bus (not shown) or over a local area network.

Exemplary embodiments described herein are illustrative, and many variations can be introduced without departing from the spirit of the disclosure or from the scope of the appended claims. For example, elements and or features of different exemplary embodiments may be combined with each other and/or substituted for each other within the scope of this disclosure and appended claims.

What is claimed is:

1. A method for correcting for magnetic field inhomogeneity in a breast MRI, comprising:
acquiring a magnetic resonance (MR) image including a patient's breast;
automatically isolating a surface image of the breast within the acquired MR image based on a high contrast between the breast and surrounding air;
generating a multiplicative field that transforms the isolated surface image into a uniform intensity; and
applying the generated multiplicative field to the entire acquired MR image to produce a corrected image.

2. The method of claim 1, further comprising:
segmenting the breast from the corrected image; and
automatically identifying one or more regions of suspicion from the segmented breast.

3. The method of claim 2, further comprising:
determining which of the identified regions of suspicion are false positives and which are actual lesions; and
presenting the location of the determined actual lesions to a user.

4. The method of claim 1, wherein the acquired MR image is a dynamic contrast enhanced MRI including a pre-contrast MR image and a sequence of post-contrast MR images acquired at a regular interval of time after administration of a magnetic contrast agent.

5. The method of claim 2, wherein:
the acquired MR image is a dynamic contrast enhanced MRI including a pre-contrast MR image and a sequence of post-contrast MR images acquired at a regular interval of time after administration of a magnetic contrast agent; and
the automatic identification of the regions of suspicion includes identifying the regions of suspicion based on an absorption and washout profile observed from the sequence of post-contrast MR images.

6. The method of claim 5, wherein one or more of the identified regions of interest are automatically characterized according to a BIRADS classification based on the absorption and washout profile for the respective identified region of suspicion observed from the sequence of post-contrast MR images.

7. The method of claim 1, wherein the surface image of the breast comprises a set of image voxels of the acquired MR image that are in contact with the surrounding air.

8. The method of claim 7, wherein the surface image of the breast further comprises image voxels of the acquired MR image that are within a predetermined depth from the image voxels of the acquired MR image that are in contact with the surrounding air.

9. The method of claim 1, wherein the surface image of the breast is an image of the breast skin.

10. A method for automatically detecting breast lesions, comprising:
receiving a dynamic contrast enhanced magnetic resonance image (DCE-MRI) including a patient's breast;
automatically isolating a surface image of the breast within the received DCE-MRI based on a high contrast between the breast and surrounding air; and
producing a corrected image from the DCE-MRT based on an intensity distribution of the isolated surface image.

11. The method of claim 10, wherein the production of the corrected image includes:
generating a multiplicative field that transforms the isolated surface image into a uniform intensity; and
applying the generated multiplicative field to the entire acquired MR image to produce a corrected image.

12. The method of claim 10, further comprising:
segmenting the breast from the corrected image;
automatically identifying one or more regions of suspicion from the segmented breast;
determining which of the identified regions of suspicion are false positives and which are actual lesions; and
presenting the location of the determined actual lesions to a user.

13. The method of claim 10, wherein the surface image of the breast comprises a set of image voxels of the acquired MR image that are in contact with the surrounding air.

14. The method of claim 13, wherein the surface image of the breast further comprises image voxels of the acquired MR image that are within a predetermined depth from the image voxels of the acquired MR image that are in contact with the surrounding air.

15. The method of claim 10, wherein the surface image of the breast is an image of the breast skin.

16. A computer system comprising:
a processor; and
a program storage device readable by the computer system, embodying a program of instructions executable by the processor to perform method steps for correcting for magnetic field inhomogeneity in a breast MRI, the method comprising:
acquiring a magnetic resonance (MR) image including a patient's breast;
automatically isolating a surface image of the breast within the acquired MR image based on a high contrast between the breast and surrounding air;
generating a multiplicative field that transforms the isolated surface image into a uniform intensity; and
applying the generated multiplicative field to the entire acquired MR image to produce a corrected image.

17. The computer system of claim 16, the method further comprising:
segmenting the breast from the corrected image;
automatically identifying one or more regions of suspicion from the segmented breast;
determining which of the identified regions of suspicion are false positives and which are actual lesions; and
presenting the location of the determined actual lesions to a user.

18. The computer system of claim 16, wherein the surface image of the breast comprises a set of image voxels of the acquired MR image that are in contact with the surrounding air.

19. The computer system of claim 18, wherein the surface image of the breast further comprises image voxels of the acquired MR image that are within a predetermined depth from the image voxels of the acquired MR image that are in contact with the surrounding air.

20. The computer system of claim 16, wherein the surface image of the breast is an image of the breast skin.
